(19) United States
(12) Patent Application Publication
(10) Pub. No.: US 2010/0252052 A1
(43) Pub. Date: Oct. 7, 2010

(54) Sexual Stimulation Device

(76) Inventors: Janice Kettles, Calgary (CA); Andrew Hicks, Canmore (CA)

Correspondence Address: HICKS & ASSOCIATES, RAVEN'S COURT, 709 MAIN STREET, SUITE 300, CANMORE, AB, T1W-2B2, CA

(21) Appl. No.: 12/751,009

(22) Filed: Mar. 31, 2010

Related U.S. Application Data
(60) Provisional application No. 61/166,291, filed on Apr. 3, 2009.

(51) Int. Cl. A61F 6/04; A61H 1/00; B28B 1/38

(52) U.S. Cl. 128/844; 601/46; 206/69; 264/301

(57) ABSTRACT

This application describes a condom having a clitoral stimulation system (CSS) integrated to the outer surface of the condom or as a separate system that may be selectively attached to the condom.

### RELATED APPLICATIONS

This application claims the benefit of priority under 35 U.S.C. §119 of U.S. Provisional Patent Application No. 61/166,291 filed Apr. 3, 2009.

### FIELD OF THE INVENTION

This application describes a condom having a clitoral stimulation system integrated to the outer surface of the condom or as a separate system that may be selectively attached to the condom.

### BACKGROUND OF THE INVENTION

As is known, for many couples during sexual intercourse, the penis does not readily stimulate the female clitoris due to the alignment between the penis and clitoris. As a result, many women find it hard to achieve orgasm, thus leading to frustration for both male and female partners. In particular, clitoral stimulation is often difficult without some form of auxiliary stimulation beyond the regular thrusting motions of sexual intercourse.

While many stimulation devices exist for the auxiliary stimulation of the clitoris (eg. vibrators and various “clit ticklers” such as a “penis ring” or “clit flicker”), there continues to be a need for improved sexual stimulation devices that provide unique functionality for clitoral stimulation. For example, there are many forms of vibrators and dildos that include various functionalities for clitoral stimulation. Such devices are manufactured from a wide variety of materials having various surface properties including rubber, acrylic, stainless steel, glass and various wood materials each of which may be intended or designed to promote clitoral stimulation. They can also include features such as variable speeds and pulsing features and can be in various forms such as strap-on devices or double-ended devices.

“Clit ticklers” and “penis rings” include many devices that are placed over the penis that during intercourse provide a more direct stimulation of the clitoris. That is, a typical penis ring will include a ring that is placed over the shaft of the penis that includes an upwardly projecting surface that when inside the vagina comes into more direct contact with the clitoris. As a result, during intercourse, such devices will increase the degree of vibratory stimulation of the clitoris. Such devices may be passive devices that require manual manipulation to cause clitoral stimulation or may include an electrical vibratory component that automatically causes clitoral stimulation.

As is also known, condoms are well known devices used extensively for contraception as well as minimizing the risks of transmitting sexually transmitted diseases (STDs).

Condoms are also known to include various stimulatory features designed to facilitate clitoral stimulation by including surface texturing on the exterior sheath of the condom by “ribs” or other raised features that are intended to enhance vibratory motion on the clitoris.

However, while each of the above past systems, to varying degrees and in different situations may provide satisfactory results to a woman or to a woman and her partner, there continues to be need for a sexual stimulation device that provides improved stimulation of the clitoris particularly in conjunction with condom use and that likewise does not decrease male stimulation. In other words, there has been a need for a system in which condoms include improved clitoral stimulation abilities while continuing to provide the simple, convenient and low-cost features of a condom that make it appealing for use by couples.

Further still, during intercourse, partners will often engage in a variety of different positions during a love making session. Generally, such positions will be either front-to-front (“frontal”) positions where the male and female are facing one another or front-to-back (“behind”) positions where the male enters the female from behind. Similarly, the partners may engage is side positions. In each position, the position of the penis relative to the vagina and clitoris is different. Specifically for the frontal and behind positions, the position of the penis relative to the vagina and clitoris is reversed. That is, during frontal intercourse, the top surface of the penis is in closer proximity to the clitoris whereas during behind intercourse, the bottom surface of the penis is in closer proximity to the clitoris. Thus, during intercourse, different areas of the penis will be contributing to clitoral stimulation.

Again, while past condoms may provide minimal stimulation to the clitoris, there has been a need for condoms that specifically enable direct clitoral stimulation during both frontal and behind intercourse without requiring adjustment (and hence risk of breakage) of the condom during an intercourse session.

A review of the prior art reveals that such condoms have not been developed.

For example, US Patent Application 2004/0133070 describes a condom device that includes an integral electric vibrator within the main body of the condom adjacent the open end of the condom. The system is packaged in a manner similar to a standard rolled condom. After placement on an erect penis (ie by un-rolling), the device is activated by pulling on a small thread which switches on the electric vibrator. U.S. Pat. No. 6,991,600 discloses a stimulatory device that is strapped to a penis and that includes a clitoris stimulating component. US Patent Application 2007/0038019 describes a penis ring that includes a microvibrator and US Patent Application 2002/0029008, US Patent Application 2006/0247493, US Patent Application 2006/0178602 and US Patent Application 2008/0077058 each describe a small vibrator that may be attached to a penis using a ring. Finally, US Patent Application 2008/0082028 describes a vibrator that may be used by both males and females.

### SUMMARY OF THE INVENTION

In accordance with the invention, there is provided a condom comprising a body having a shaft portion and a base portion; a clitoral stimulation system (CSS) located on the shaft portion adjacent the base portion wherein the CSS is sized and positioned for clitoral stimulation during intercourse.

In different embodiments, the CSS may be in various individual or combined forms including:
a. an integrated hump on the shaft portion;
b. a resiliently flexible material extending upwardly 0.25-1.0 inches;
c. extending around 15-25% of the outer perimeter of the shaft portion;
d. a length greater than 1.0 inches;
e. including at least one auxiliary projection extending outwardly from the integrated hump;
f. including a plurality of outwardly projecting resiliently flexible fins.

In other embodiments, the body includes a CSS pocket for insertion of a separate CSS into the CSS pocket. The separate CSS may be a resiliently flexible hump having an inner concave surface corresponding in radius to the typical outer radius of an erect penis. The separate CSS may be a vibrator and may have an inner concave surface corresponding in radius to the typical outer radius of an erect penis and/or an outer convex surface adapted for tight frictional engagement with the inner surfaces of the pocket.

In another embodiment, the body includes at least two CSS pockets for insertion of separate CSS's into each CSS pocket. The body may include two CSS pockets positioned for frontal and behind intercourse positions and/or for G-spot stimulation.

In accordance with another embodiment, the invention provides a method of manufacturing a condom having a CSS comprising the step of solution casting a condom on a mold within a liquid solution wherein at least one CSS is operatively connected to the condom either before or after the mold is solution cast within the liquid solution. In another embodiment of the method, the CSS includes at least one CSS pocket and the mold includes a first mold and at least one pocket mold and wherein the at least one pocket mold is operatively connected to the condom after solution casting and the at least one pocket mold is subsequently cast with the liquid solution. In one embodiment, the CSS is operatively connected to the condom after solution casting and the CSS is subsequently cast with the liquid solution. The mold may be solution cast in latex rubber or polyurethane.

In yet another embodiment, the invention provides a condom kit comprising: at least one condom having a body having a shaft portion, a base portion and at least one clitoral stimulation system (CSS) pocket, the at least one CSS pocket for securing at least one CSS within each CSS pocket; condom packaging for operatively and separately containing each at least one condom; at least one separate CSS for operative insertion within each at least one CSS pocket and wherein each CSS is sized and positioned for clitoral or vaginal stimulation during intercourse; and, packaging for containing each of the at least one condom, condom packaging and at least one separate CSS.

### BRIEF DESCRIPTION OF THE DRAWINGS

The invention is described with reference to the accompanying figures in which:

FIG. 1 is a cross-sectional drawing of a condom in accordance with a first embodiment of the invention having an integral clitoral stimulation system (CSS);

FIG. 1A is an end view of the condom of FIG. 1;

FIG. 1B is an end view of the condom in accordance with the invention having an alternate CSS;

FIG. 1C is an end view of a the condom in accordance with the invention having an alternate CSS;

FIG. 2 is a side view of a condom in accordance with one embodiment of the invention having a CSS pocket;

FIG. 2A is plan view of vibrator in accordance with one embodiment of the invention;

FIG. 2B is an end view of a vibrator in accordance with one embodiment of the invention;

FIG. 3 is a schematic diagram showing one method of manufacture in accordance with an embodiment of the invention;

FIG. 4 is a schematic diagram showing one method of manufacture in accordance with an alternate embodiment of the invention;

FIG. 5 is a schematic diagram showing one method of manufacture in accordance with another embodiment of the invention; and

FIG. 6 is a cross-sectional drawing of a condom in accordance with a second embodiment of the invention having a second integral clitoral stimulation system (CSS).

### DETAILED DESCRIPTION OF THE INVENTION

### Overview

With reference to the figures, a condom having improved clitoral stimulation features is provided. As is known, the average erect human penis size is approximately 5.1-5.9 inches with an average erect diameter of 1.5 inches.

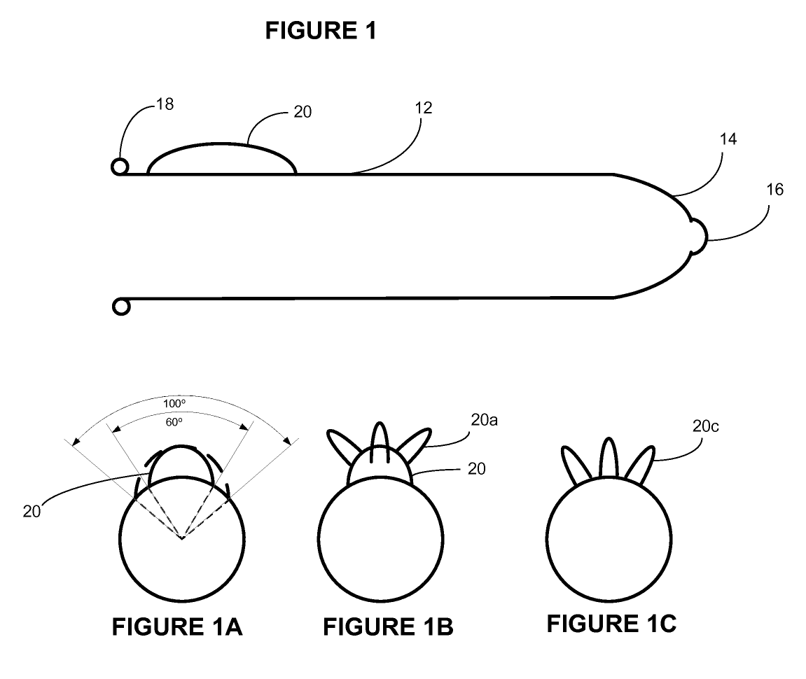

As shown in FIG. 1, a condom 10 in accordance with the invention includes a shaft portion 12, head portion 14, receptacle 16, shaft base portion 18 and clitoral stimulation portion (CSP) 20. The CSP 20 is positioned near or adjacent the shaft base portion 18 on the outer surface of the shaft portion. As will be explained in greater detail below, the CSP may be in manufactured in a number of forms so as to provide variety and flexibility in the manner in the invention can be designed and used. The condom 10 is generally fabricated from materials including latex rubber and polyurethane as known to those skilled in the art.

In its most basic form, the CSP is a simple solid or semi-solid hump 20 as shown in FIG. 1A. The hump is positioned on the outer surface of the condom and extends forwards from the shaft base portion approximately 1-2 inches and outwardly by approximately 0.25 to 1.0 inches. The hump 20 is integral with the shaft portion and will typically extend 60-100 degrees around the perimeter of base portion of the shaft. A larger hump is shown in dotted lines in FIG. 1A. In the most basic embodiment, the hump is a resiliently flexible extension having sufficient rigidity and flexibility so as to provide clitoral stimulation during intercourse.

As shown in FIGS. 1B and 1C, alternate embodiments of the CSP are shown in which variations on the basic hump are shown. For example, as shown in FIG. 1B, further projections 20a may be provided on the hump 20 in a variety of lengths and configurations so as to provide different forms of clitoral stimulation and variety to the consumer. Alternatively, as shown in FIG. 10, the hump may be a number of different projections 20c attached directly to the shaft portion at different angles and at a variety of different lengths. However, in each case, the CSP generally has a “footprint” of approximately at least ⅝″ (width), ¼″ (height) and 1″ (length). Larger “footprints” as shown in FIGS. 1A, 1B and 10 may be possible.

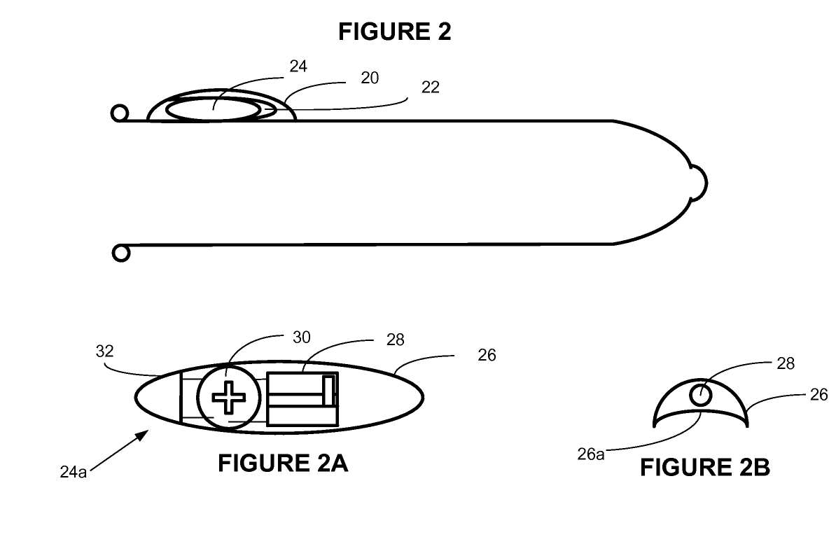

As shown in FIG. 2, a further embodiment of the invention is shown in which the “hump” is formed with a pocket 22 for placement of a CSP insert 24 within the pocket. In this case, the insert 24 can be a variety of CSP devices having a variety of different sizes or shapes. Due to the resiliently flexible construction of a condom, a variety of different sized CSP inserts can be placed within the pocket 22.

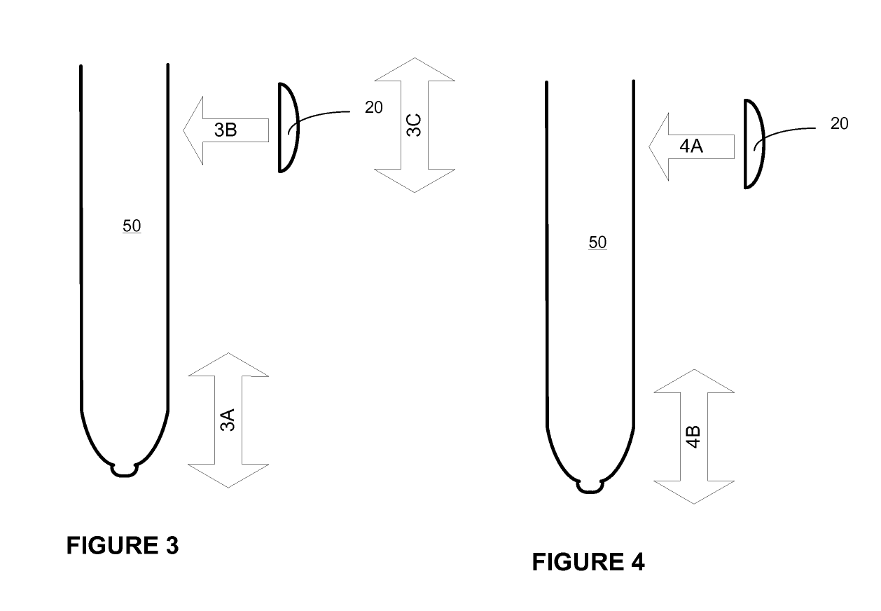

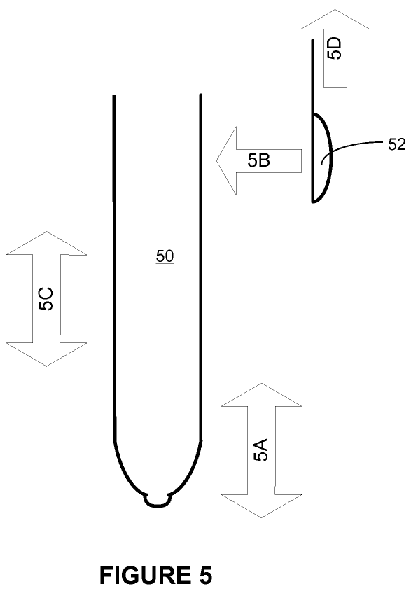

In one embodiment, the CSP insert 24 is a vibrator device 24a as shown in FIG. 2A. The vibrator 24a would generally include a case 26 containing a small vibrating motor 28 and power supply 30 and switch 32. As shown, the vibrator may be contoured as shown in FIG. 3A so as to comfortably fit in the pocket and against a penis. That is, the inner surface 26a of the vibrator will have a corresponding curvature to that of a typical erect penis. The vibrating motor, power supply and switch may be encased in a typical or known flexible rubber materials so as to provide suitable levels of comfort to the users.

Importantly, these embodiments would enable the user to insert and remove the CSP devices into the condom for washing and re-use whilst the condom with a pocket could be disposed of in the usual way.

### Manufacture and Packaging

Various embodiments of the condom may be manufactured utilizing the following methodologies.

In one embodiment as shown in FIG. 3, the condom is cast on a mold 50 by vertically dipping the mold in a liquid solution of latex or polyurethane, removing the coated mold and allowing the resulting layer of liquid solution to cure or partially cure on the mold (step 3A). The CSS is then secured to the cured or partially-cured shaft formed in the first step (step 3B) and the mold is dipped in the liquid solution a second time (step 3C). The condom is then removed from the mold by rolling the cured material from the mold or by air-injection. After removal, the condom is placed in suitable packaging.

In another embodiment as shown in FIG. 4, the CSS is secured to the mold prior to dipping (step 4A) and a single dip (Step 4B) in the liquid solution would be utilized.

In an embodiment incorporating a pocket 22, a two part mold may be utilized including a first mold 50 and pocket mold 52. In this case, the main shaft of the condom may be cast initially without the pocket mold (Step 5a). After curing or partial curing of the first cast layer, the pocket mold may be placed against the first cast layer (Step 5B) and dipped a second time (Step 5C). The pocket mold 52 is then removed (Step 5D). The second dip may be from the same direction such that the condom is comprised of two dip cast layers or from an alternate direction such that only the pocket mold 52 is immersed in the liquid solution so as to prevent over thickening of the condom at the head end of the condom. This may be desirable so as to maintain sensitivity to the male during use. In another embodiment, the pocket mold or CSS is sprayed with liquid solution from an alternate direction in order to cover only the pocket mold or CSS.

After the condom has fully cured and the pocket mold has been removed, the condom is packaged.

Depending on the size of the CSS, the condom may not be readily rolled off the mold and hence, the condom may not be packaged as a conventional condom would be as a small square package with a “ring” of rolled rubber or latex. In these embodiments, it may be necessary to fold or package the condom in an alternate package which would alter the manner in which the condom was placed on the erect penis prior to intercourse. Accordingly, in some embodiments, a film of lubricant may be added to the inner surface of the condom to assist in sliding the condom onto the penis instead of rolling it onto the penis.

In the case of the pocket mold, in most cases the condom could be rolled and packaged in the conventional manner.

Depending on the embodiment, the condom may be packaged as a typical single use condom, as a “kit” in which a number of different condoms having different CSS's are included, or as separate components that can be purchased separately.

More specifically, a single-use condom would typically include an integrated CSS. The condom would be packaged as single condom or as a multiple package (individual condom packages connected together) and each condom would be disposed of after use.

A kit would generally include one or more pocket condoms with one or more separate CSS's. In this case, the kit could include different CSS's that could be individually inserted into a pocket condom or the kit could include one or more condoms with an integrated CSS. Accordingly, the users could “try-out” different CSS's to determine which type of CSS they enjoyed the most. In this case, the kit could include the vibrator option.

In another embodiment, the purchasers simply purchase the desired CSS's and pocket condoms in any desired combination. For example, the users may determine that the vibrator/pocket condom combination is their favorite combination in which case, they would simply purchase one or more single use pocket condoms and re-use the vibrator in each condom. Alternatively, the users may decide to maintain a collection of different “passive” CSS designs that are selectively used in individually packaged pocket condoms.

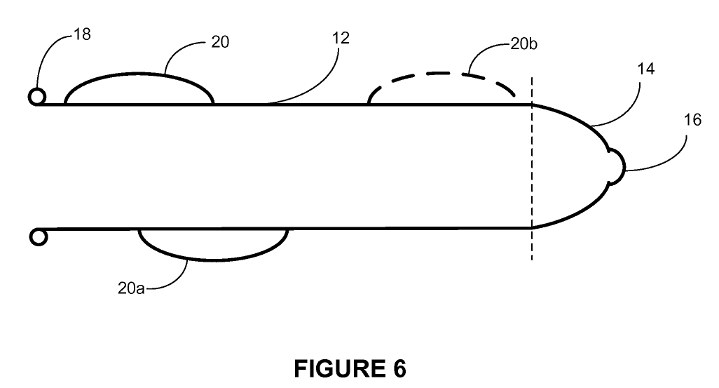

As shown in FIG. 6, a further embodiment of the invention is described in which at least one additional pocket or CSS 20a (20b) is incorporated at a separate location (circumferential and/or longitudinal) position on the condom specifically for frontal and behind intercourse positions. As above, the CSS may be integrated with the condom or may be inserted into pockets. In yet further embodiments, other pockets or CSS's may be incorporated at positions nearer the head of the penis to facilitate stimulation of the G-spot believed to be located at a deeper location within the vagina. Thus, in accordance with the invention, couples may be given the opportunity to customize a condom in accordance with their own experiences with different variations of the invention. For example, some couples may only choose to use a single CSS condom while others, as a result of their position practices, may utilize a dual CSS condom. Further still, for those couples may choose a further embodiment that includes a G-spot stimulation system such as 20b.

The dotted line in FIG. 6 shows yet a further embodiment of the invention in which the head of the condom is removed. This embodiment, while clearly no longer a condom suitable for birth and/or STD control may be appealing to couples simply for the female stimulation functionality.

While the invention has been described by the foregoing description and the figures, it is understood that various modifications may be made without departing from the scope of the invention.

1. A condom comprising:
a) a body having a shaft portion and a base portion;
b) an clitoral stimulation system (CSS) located on the shaft portion adjacent the base portion wherein the CSS is sized and positioned for clitoral stimulation during intercourse.

2. A condom as in claim 1 wherein the CSS is an integrated hump on the shaft portion.

3. A condom as in claim 2 wherein the integrated hump is a resiliently flexible material extending upwardly 0.25-1.0 inches.

4. A condom as in claim 2 wherein the integrated hump extends around 15-25% of the outer perimeter of the shaft portion.

5. A condom as in claim 2 wherein the integrated hump has a length greater than 1.0 inches.

6. A condom as in claim 2 wherein the integrated hump includes at least one auxiliary projection extending outwardly from the integrated hump.

7. A condom as in claim 1 wherein the CSS includes a plurality of outwardly projecting resiliently flexible fins.

8. A condom as in claim 1 wherein the body includes a CSS pocket for insertion of a separate CSS into the CSS pocket.

9. A condom as in claim 8 wherein the separate CSS is a resiliently flexible hump having an inner concave surface corresponding in radius to the typical outer radius of an erect penis.

10. A condom as in claim 8 wherein the separate CSS is a vibrator.

11. A condom as in claim 10 wherein the vibrator has an inner concave surface corresponding in radius to the typical outer radius of an erect penis.

12. A condom as in claim 11 wherein the vibrator has an outer convex surface adapted for tight frictional engagement with the inner surfaces of the pocket.

13. A condom as in claim 1 wherein the body includes at least two CSS pockets for insertion of separate CSS's into each CSS pocket.

14. A condom as in claim 13 wherein the body includes two CSS pockets positioned for frontal and behind intercourse positions.

15. A condom as in claim 13 wherein the body includes two CSS pockets wherein in at least one CSS pocket is positioned for G-spot stimulation.

16. A condom as in claim 1 in which the body of the condom does not include a head portion.

17. A method of manufacturing a condom having a CSS comprising the step of:
solution casting a condom on a mold within a liquid solution wherein at least one CSS is operatively connected to the condom either before or after the mold is solution cast within the liquid solution.

18. A method as in claim 17 wherein the CSS includes at least one CSS pocket and the mold includes a first mold and at least one pocket mold and wherein the at least one pocket mold is operatively connected to the condom after solution casting and the at least one pocket mold is subsequently cast with the liquid solution.

19. A method as in claim 17 wherein the CSS is operatively connected to the condom after solution casting and the CSS is subsequently cast with the liquid solution.

20. A method as in claim 17 wherein the mold is solution cast in latex rubber or polyurethane.

21. A condom kit comprising:
at least one condom having a body having a shaft portion, a base portion and at least one clitoral stimulation system (CSS) pocket, the at least one CSS pocket for securing at least one CSS within each CSS pocket;
condom packaging for operatively and separately containing each at least one condom;
at least one separate CSS for operative insertion within each at least one CSS pocket and wherein each CSS is sized and positioned for clitoral or vaginal stimulation during intercourse; and,
packaging for containing each of the at least one condom, condom packaging and at least one separate CSS.

22. A condom kit as in claim 21 wherein the at least one separate CSS is a vibrator.
